(19) United States
(12) Patent Application Publication
(10) Pub. No.: US 2010/0178371 A1
(43) Pub. Date: Jul. 15, 2010

(54) COMPOSITION FOR SUPRESSING APPETITE, IMPROVING TONE AND MOOD, WITH A NATURAL ANTIDEPRESSANT ACTIVITY AND WITH AN ANTIASTHENIC EFFECT

(76) Inventor: Giulia Federica Merizzi, Torino (IT)

Correspondence Address: Steinfl & Bruno, 301 N Lake Ave Ste 810, Pasadena, CA, 91101, US

(21) Appl. No.: 12/663,249

(22) PCT Filed: Jun. 3, 2008
(86) PCT No.: PCT/IB08/52154

§ 371 Date: Dec. 4, 2009

(30) Foreign Application Priority Data
Jun. 5, 2007 (IT) TO2007A000391

(51) Int. Cl. A61K 35/74; A61K 31/405; A61P 3/04; A61P 25/00

(52) U.S. Cl. 424/780; 514/419

(57) ABSTRACT

Compositions comprising L-tryptophan and/or 5-hydroxytryptophan are used for preparing a product suitable for sublingual or nasal administration which is helpful in suppressing appetite in such a manner as to promote weight loss in an individual; such compositions furthermore exhibit an antidepressant activity and an antiasthenic effect which makes them helpful in improving tone and mood and the level of attention in an individual. The compositions preferably comprise phenylethylamine in a synergistic combination which substance is contained in an algal plant extract, preferably of Klamath algae.

The present invention relates to pharmaceutical or food supplement compositions or a medical device having an antidepressant activity and antiasthenic effect and to the use thereof in suppressing appetite in such a manner as to promote weight loss and for improving tone and mood.

The problem of hunger in an overweight individual has always been the underlying factor in the failure of any diet and also results in swings in level of mood, nervous tension, a feeling of frustration; this situation does not only occur while the attempt is being made to slim, but also when maintaining weight loss, often resulting in the lost weight being regained.

A substance, serotonin, is present in our central nervous system which acts as a neurotransmitter, namely is produced selectively by a nerve ending subsequent to a specific stimulus. Serotonin has an action on some aspects of mood and sleep and an association has been identified between a deficiency of serotonin and depression. Hunger, satiety, emotional equilibrium and some food-related behaviours are all mediated by serotonin in certain regions of the hypothalamus. Chronic headaches, in particular migraines, are also the result of low serotonin levels.

L-Tryptophan is present in the plasma, both in free form and bound to plasma proteins: however, it is only the free form which is capable of passing through the blood-brain barrier in order to be converted into 5-HTP (5-hydroxytryptophan), which is the precursor of serotonin and is subsequently converted into serotonin.

The present invention firstly provides the use of a composition comprising L-tryptophan and/or 5-hydroxytryptophan for preparing a product for sublingual or nasal administration which is helpful in suppressing appetite and in promoting weight loss in an individual.

The present invention also provides the use of the above-stated composition for preparing a sublingually or nasally administrable product which has an antidepressant and antiasthenic activity and is thus helpful in improving not only tone and mood, but also the level of attention and mental energy.

The sublingual route of administration is preferred because it permits faster absorption of the substance tryptophan in free form and then through the blood-brain barrier.

To this end, the product provided by the invention is preferably formulated in liquid form for spray application (oral spray); however, other administration forms, such as for example paper, film or soluble tablets for oral/sublingual application, are envisaged.

Preferably used sources of tryptophan or 5-HTP are plant extracts with an elevated 5-HTP content, especially extracts from leguminous plants and in particular extracts from the African plant Griffonia simplicifolia.

Griffonia simplicifolia extracts of a titrated 5-HTP strength with a 5-HTP content generally of between 15% and 25% are commercially available and may be used for the purposes of the invention.

In a preferred embodiment, the product provided by the invention furthermore comprises a source of phenylethylamine (PEA), preferably composed of an extract of blue-green algae, preferably of a Klamath algae extract. It is known that blue-green microalgae contain a significant quantity of phycocyanins, together with variable amounts of phenylethylamine. In particular, Klamath algae is the only food hitherto known to contain significant quantities of phenylethylamine, an amino acid naturally produced by our brain in states of euphoria and joy which directly assists in raising the level of freely circulating dopamine, so increasing dopaminergic transmission.

In the brain, the action of PEA is based on the fact that it has a greater affinity than does dopamine itself for the mechanism for reuptake of dopamine into presynaptic vesicles. This means that, once it reaches the brain, it is captured by the presynaptic vesicles and occupies the site normally occupied by dopamine. This results in an increase in the level of freely circulating dopamine in the presynaptic terminals and in a greater concentration of diffuse dopamine in the synaptic gaps, so strengthening dopaminergic transmission.

This capability of modulating dopaminergic transmission means that PEA has properties of interest for alleviating depression and attention disorders and for improving concentration and mood. For the purposes of the present invention, extracts of Klamath algae are used in compositions helpful in regulating appetite, preferably in a synergistic combination with L-tryptophan and/or 5-HTP.

The present invention accordingly also provides the use of extracts of blue-green algae, particularly of Klamath algae, preferably combined with L-tryptophan and/or 5-HTP, for preparing a product intended for oral, and in particular sublingual, administration which is helpful in suppressing appetite and in promoting weight loss in an individual.

As has been stated, the preferred embodiment envisages combining L-tryptophan and/or 5-HTP with extracts of Klamath algae. Such extracts are commercially available. In particular, commercial extracts typically containing from 0.5% to 2% by weight of phenylethylamine may be used.

The composition according to the invention may moreover comprise further plant extracts, preferably selected from among extracts of Centella asiatica, guarana, Taraxacum, artichoke, Gingko biloba which do or do not comprise biflavones and mixtures thereof; the above-stated extracts being preferably in phytosomal form (complexed with phospholipids).

In the case of a liquid product usable as a sublingual spray, the formulation comprises the above-stated extracts dissolved in an aqueous vehicle, optionally including a pharmaceutically acceptable solvent.

A formulation according to the invention typically contains, relative to 100 ml of formulation:
L-tryptophan or 5-HTP from 100 to 20000 mg, preferably from 1000 to 5000 mg
phenylethylamine from 5 to 2000 mg, preferably from 10 to 300 mg.

It will be understood that the composition may furthermore contain preservatives, such as for example methyl hydroxybenzoate (non-sodium) or propyl hydroxybenzoate.

The compositions according to the invention may be offered for sale as a drug, food supplement or medical device.

The usefulness of the above-described compositions in suppressing appetite was tested by means of the following study.

### Testing

The study was carried out using a double blind protocol on two treatment groups each comprising 15 patients. In a first study, the active product used was a product having the following formulation:
30 ml bottle:
Griffonia simplicifolia titrated to 25%: 3 g dry extract
guarana: 350 mg
Centella leaf extract: 0.9 g (3 g per 100 ml)
Taraxacum leaf extract: 0.9 g (3 g per 100 ml)
artichoke leaf extract: 0.75 g (2.5 g per 100 ml)
fructose syrup or sorbitol syrup: 30%
purified water: q.s.
preservatives: methyl hydroxybenzoate (non-sodium) or propyl hydroxybenzoate: 0.1%.

The patients were recruited in a screening visit (time 0) and were randomly assigned to one of the two groups. A first check (time 1) was carried out after a fortnight and a second check (time 2) thirty days from time 0. Over this period, the patients followed a low-calorie diet and kept a diary recording subjective symptoms relating to feelings of hunger, anxiety and to the difficulty of observing the proposed diet. To this end, a visual analogue scale with values from 1 to 10 was used to indicate the intensity of these symptoms.

The product was administered sublingually with three sprays at a time, every three hours, for a maximum of five times per day (h 07:00, 10:00, 13:00, 16:00, 19:00). A selected subgroup was provided who received an evening dose (h 22:00) to control night-time awakening. Each spray administers a dose of 0.3 ml (containing approx. 30 mg of natural extract of Griffonia simplicifolia, titrated to a tryptophan content of 25%), giving an overall daily dose of 450 mg (equivalent to five daily administrations each comprising three sprays); for the above-stated subgroup, the overall daily dose was 540 mg, in six administrations (always three sprays each).

Treatment lasted thirty days and each patient received detailed information at the start of testing on the nature of the study, its duration and the methods used.

Each item of data, recorded on suitable clinical cards and analysed, revealed the following results: the group having taken the product according to the invention did indeed exhibit greater control of the feeling of hunger, with remission of the symptom for up to 2.5 hours from taking (with a mean value of 1.38 hours); the group treated with the placebo did not exhibit any change, except for three test subjects and for a duration of no more than 30 minutes (with a mean value of 19.8 minutes).

In the subgroup of eight test subjects who had reported the problem of night-time awakening associated with hunger, the four who had taken the product according to the invention had all significantly reduced the frequency of awakening after a fortnight's treatment; of the other four, who had taken the placebo, only one reported a reduction in this problem.

As a result of the better control of the feeling of hunger in the group treated with the product, on completion of the study, a greater reduction in body weight was observed relative to the group treated with placebo. Administration of the product had also brought about a significant reduction in body weight in three patients suffering from type 2 diabetes.

A second study was carried out using the same formulation shown above and additionally containing:
Klamath extract: 2000 mg in 30 ml.

In this case, analysis of the results confirmed the positive outcome shown above with a mean value for remission of the symptom of a feeling of hunger of 3 hours.

In both studies, the test subjects who were taking the active treatment reported a distinct improvement in mood combined with a better level of attention and “mental energy” with an antiasthenic effect resulting from taking the product.

1-18. (canceled)

19. A method comprising
administering to an individual a composition comprising an extract of Griffonia simplicifolia, and/or a Klamath algae extract,
wherein the extract of Griffonia simplicifolia comprises L-tryptophan and/or 5-hydroxytryptophan and
wherein the administering is performed by oral spray and results in at least one of suppressing appetite, promoting weight loss, and improving the level of attention, tone and/or mood in the individual.

20. The method according to claim 19, wherein said composition comprising the Klamath algae extract comprises phenylethylamine.

21. The method according to claim 19, wherein said composition comprises L-tryptophan and/or 5-hydroxytryptophan in a quantity of 100 mg to 20000 mg/100 ml.

22. The method according to claim 19, wherein said composition comprises phenylethylamine in a quantity of between 5 mg and 2000 mg/100 ml.

23. The method according to claim 21, wherein said composition comprises phenylethylamine in a quantity of between 5 mg and 2000 mg/100 ml.

24. The method according to claim 19, wherein said composition furthermore comprises one or more plant extracts selected from the group consisting of guarana extract, artichoke extract, Centella extract, Taraxacum extract, Gingko biloba extract including or not including biflavones, preferably in phytosomal form, and mixtures thereof.

25. The method according to claim 19, wherein said composition comprises L-tryptophan and/or 5-hydroxytryptophan in a quantity of 1000 to 5000 mg/100 ml.

26. The method according to claim 19, wherein said composition comprises phenylethylamine in a quantity of 10 to 300 mg/100 ml.

27. The method according to claim 25, wherein said composition comprises phenylethylamine in a quantity of 10 to 300 mg/100 ml.
